(19) United States
(12) Patent Application Publication
(10) Pub. No.: US 2023/0218825 A1
(43) Pub. Date: Jul. 13, 2023

(54) AUTO-INJECTOR ASSEMBLY

(71) Applicant: BIOCON BIOLOGICS LIMITED, Karnataka (IN)

(72) Inventors: Jeevan Maddur SHANKARSETTY, Karnataka (IN); Shanthan JANGILI, Telangana (IN); Ayanur Nadig Chetan RAO, Karnataka (IN); Shreehas Pradeep TAMBE, Karnataka (IN); Anu Kumar Haranahalli SADASHIVA, Mysuru (IN)

(73) Assignee: BIOCON BIOLOGICS LIMITED, Karnataka (IN)

(21) Appl. No.: 17/922,713

(22) PCT Filed: May 14, 2021
(86) PCT No.: PCT/IN2021/050464

§ 371 Date: Nov. 1, 2022

(30) Foreign Application Priority Data
May 15, 2020 (IN) 202041020641

(51) Int. Cl. A61M 5/20; A61M 5/32; A61M 5/315

(52) U.S. Cl. CPC A61M 5/2033; A61M 5/326; A61M 5/31551; A61M 2005/2013; A61M 2005/3267

(57) ABSTRACT

AUTO-INJECTOR ASSEMBLY The auto-injector assembly (100) includes a housing (102), a syringe assembly (202) to store a predetermined volume of a drug to be administered, a needle guard (212) to surround the syringe assembly (202) such that a portion of the needle guard (212) is adapted to extend out of the housing (102) in an initial state and to retract into the housing (102) when the portion is pressed on to an injection site, a spring holder (218) to hold a dose delivery spring (216) in a compressed state, locking unit (220) to keep the spring holder (218) in a locked state, and a plunger (222) engaged with the spring holder (218) and adapted to move towards the syringe assembly (202) when the spring holder (218) is unlocked. A position of engagement of the plunger 222) with the spring holder (218) is adapted to change for controlling a travelling distance of the plunger (222) within the housing (102) based on the predetermined volume of the drug in the syringe assembly (202).

### FIELD OF THE INVENTION

The present disclosure relates to auto-injectors and more particularly, relates to an auto-injector assembly having a plunger and adapted to operate to deliver different volumes of a drug into an injection site.

### BACKGROUND

Auto-injectors are widely used for direct delivery of drugs into a patient's body. Nowadays, there are a plethora of auto-injectors available in the market. The existing auto-injectors are mainly capable of delivering a predetermined dose of medication to the patient. Typically, the conventional auto-injector is adapted to deliver a predetermined dosage of a drug to a patient.

As is generally known, each drug requires to be delivered in different volumes to a patient. The volume may also change depending on the patient, for example, based on a condition and the age of the patient. Similarly, there are multiple other factors contributing in determining a dosage of a drug to be injected into the patient's body. Therefore, same drug may have to be delivered in different volumes to different patients for effective treatment. However, a manufacturer is required to manufacture a specific auto-injector for a predefined volume of drug to be administered.

Therefore, delivering a specific volume of a drug would require construction of a particular auto-injector that is capable of delivering the predetermined dosage to the patient. Owing to such constructional limitation, for dosage of different volumes, multiple auto-injectors are required. Therefore, for a consumer, it is inconvenient to purchase, operate, and maintain multiple auto-injectors. On the other hand, a drug manufacturer also has to manufacture multiple auto-injectors for different drugs that require different dosage settings. For example, in terms of infrastructure of the manufacturing plant, the manufacturer may have to make significant effort and investment.

There are a few device manufacturers offering changes to constituent components of the auto-injectors allowing for setting variable dose of drugs for delivery. However, the manufacturing of such auto-injectors demands significant assembly-line changes in the manufacturing plant. Consequently, an overall operational cost of the auto-injectors is also high.

### SUMMARY

This summary is provided to introduce a selection of concepts, in a simplified format, that are further described in the detailed description of the invention. This summary is neither intended to identify key or essential inventive concepts of the invention and nor is it intended for determining the scope of the invention.

In an embodiment of the present disclosure, an auto-injector assembly is disclosed. The auto-injector assembly includes a housing having a proximal end and a distal end, a syringe assembly disposed adjacent to the proximal end of the housing and adapted to store a predetermined volume of a drug to be administered through a needle, a needle guard disposed to surround the syringe assembly such that a portion of the needle guard is adapted to extend out of the housing in an initial state and to retract into the housing when the portion is pressed on to an injection site, a spring holder disposed adjacent to the distal end of the housing and adapted to hold a dose delivery spring in a compressed state, a locking unit disposed inside the spring holder and adapted to keep the spring holder in a locked state with the housing, and a plunger engaged with the spring holder and adapted to move towards the syringe assembly when the spring holder is unlocked. A position of engagement of the plunger with the spring holder is adapted to change for controlling a travelling distance of the plunger within the housing based on the predetermined volume of the drug in the syringe assembly. The needle guard, when being retracted, is adapted to push the locking unit further into the spring holder, unlocking the spring holder from the housing such that the plunger is moved towards the syringe assembly for administering the drug into the injection site.

To further clarify the advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates a perspective view of an auto-injector assembly, according to an embodiment of the present disclosure;

FIG. 2 illustrates an exploded view of the auto-injector assembly depicting various constituent components, according to an embodiment of the present disclosure;

FIG. 3 illustrates another exploded view of the auto-injector assembly, according to an embodiment of the present disclosure;

FIG. 4 illustrates a sectional view of the auto-injector assembly depicting various constituent components, according to an embodiment of the present disclosure;

FIG. 5 illustrates a sectional view of a portion of the auto-injector assembly depicting locking of a spring holder with a housing, according to an embodiment of the present disclosure;

FIG. 6 illustrates a sectional view of the auto-injector assembly depicting unlocking of the spring holder owing to movement of a needle guard, according to an embodiment of the present disclosure;

FIG. 7A illustrates a sectional view of the auto-injector assembly depicting position of constituent components in an initial state, according to an embodiment of the present disclosure;

FIG. 7B illustrates a sectional view of the auto-injector assembly depicting position of constituent components after drug is administered, according to an embodiment of the present disclosure;

FIG. 8A illustrates positioning of the needle guard with respect to a syringe holder in the initial state, according to an embodiment of the present disclosure;

FIG. 8B illustrates positioning of the needle guard with respect to the syringe holder after the drug is administered, according to an embodiment of the present disclosure; and

FIG. 9 illustrates an example depicting usage of the auto-injector assembly by a user, according to an embodiment of the present disclosure.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DETAILED DESCRIPTION OF FIGURES

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. Unless otherwise defined, all technical and scientific terms used herein have the same meaning as commonly understood by one of ordinary skilled in the art to which this invention belongs. The system, methods, and examples provided herein are illustrative only and not intended to be limiting.

For example, the term “some” as used herein may be understood as “none” or “one” or “more than one” or “all.” Therefore, the terms “none,” “one,” “more than one,” “more than one, but not all” or “all” would fall under the definition of “some.” It should be appreciated by a person skilled in the art that the terminology and structure employed herein is for describing, teaching and illuminating some embodiments and their specific features and elements and therefore, should not be construed to limit, restrict or reduce the spirit and scope of the present disclosure in any way.

For example, any terms used herein such as, “includes,” “comprises,” “has,” “consists,” and similar grammatical variants do not specify an exact limitation or restriction, and certainly do not exclude the possible addition of one or more features or elements, unless otherwise stated. Further, such terms must not be taken to exclude the possible removal of one or more of the listed features and elements, unless otherwise stated, for example, by using the limiting language including, but not limited to, “must comprise” or “needs to include.”

Whether or not a certain feature or element was limited to being used only once, it may still be referred to as “one or more features” or “one or more elements” or “at least one feature” or “at least one element.” Furthermore, the use of the terms “one or more” or “at least one” feature or element do not preclude there being none of that feature or element, unless otherwise specified by limiting language including, but not limited to, “there needs to be one or more . . . ” or “one or more element is required.”

Unless otherwise defined, all terms and especially any technical and/or scientific terms, used herein may be taken to have the same meaning as commonly understood by a person ordinarily skilled in the art.

Reference is made herein to some “embodiments.” It should be understood that an embodiment is an example of a possible implementation of any features and/or elements of the present disclosure. Some embodiments have been described for the purpose of explaining one or more of the potential ways in which the specific features and/or elements of the proposed disclosure fulfil the requirements of uniqueness, utility, and non-obviousness.

Use of the phrases and/or terms including, but not limited to, “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or other variants thereof do not necessarily refer to the same embodiments. Unless otherwise specified, one or more particular features and/or elements described in connection with one or more embodiments may be found in one embodiment, or may be found in more than one embodiment, or may be found in all embodiments, or may be found in no embodiments. Although one or more features and/or elements may be described herein in the context of only a single embodiment, or in the context of more than one embodiment, or in the context of all embodiments, the features and/or elements may instead be provided separately or in any appropriate combination or not at all. Conversely, any features and/or elements described in the context of separate embodiments may alternatively be realized as existing together in the context of a single embodiment.

Any particular and all details set forth herein are used in the context of some embodiments and therefore should not necessarily be taken as limiting factors to the proposed disclosure.

The present disclosure relates to an auto-injector assembly having a plunger and capable of administering different volumes of a drug. The position of the plunger can be adjusted to vary the travelling distance within a housing, based on different volumes of a drug to be delivered to a patient. The position has to be adjusted such that the plunger can travel the required distance to push the drug out of a syringe assembly. The plunger is adapted to be engaged at different positions with a spring holder based on dosage requirement for varying the travelling distance of the plunger within the housing. This adjustment of the position of the plunger happens during the manufacturing stage only, for example, by the manufacturer, depending on a predefined volume of the drug to be administered by a particular device. Therefore, the user is not required to make any adjustment to the device and can directly use the device.

The auto-injector assembly may also include one or more stoppers on an inner surface of the housing for engaging the spring holder, restricting the movement of the plunger for administering the drug, unless the user manually activates the device. Only when the spring holder is disengaged from the stoppers, which happens upon manual activation by the user, the plunger is adapted to move to push the drug into the injection site.

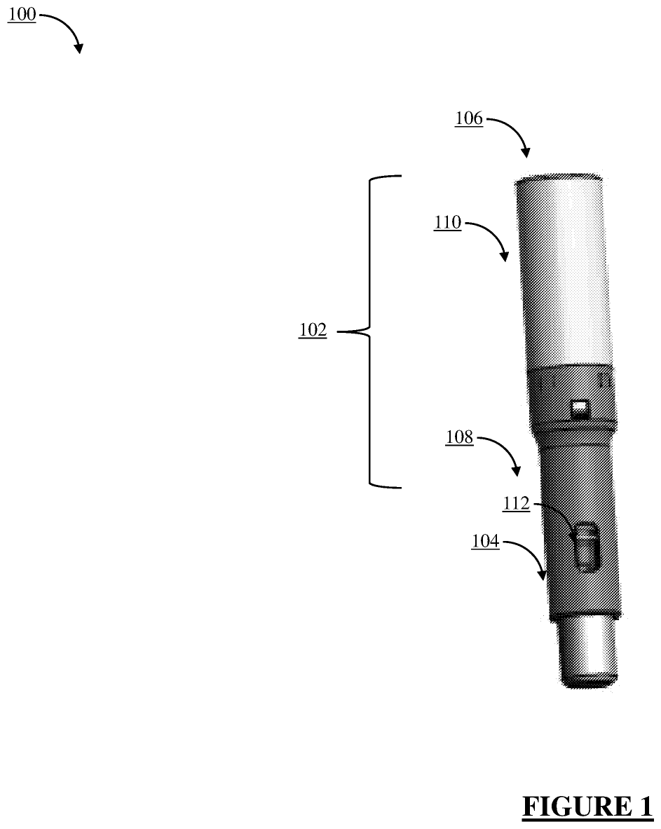

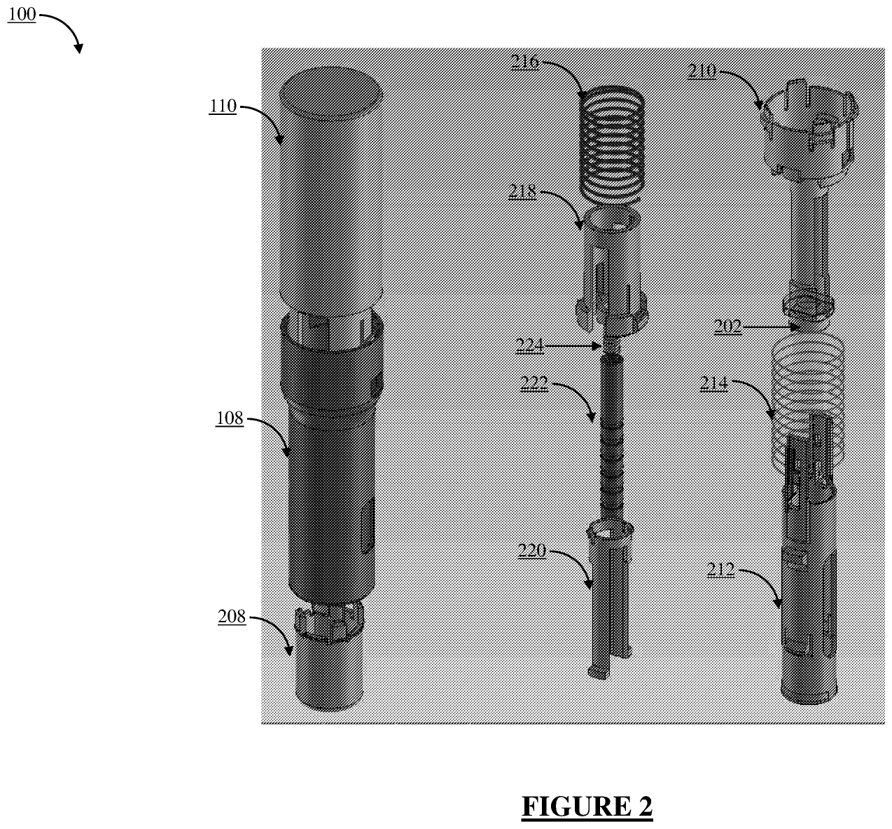

For the sake of clarity, the first digit of a reference numeral of each component of the present disclosure is indicative of the Figure number, in which the corresponding component is shown. For example, reference numerals starting with digit “1” are shown at least in FIG. 1. Similarly, reference numerals starting with digit “2” are shown at least in FIG. 2.

Embodiments of the present invention will be described below in detail with reference to the accompanying drawings.

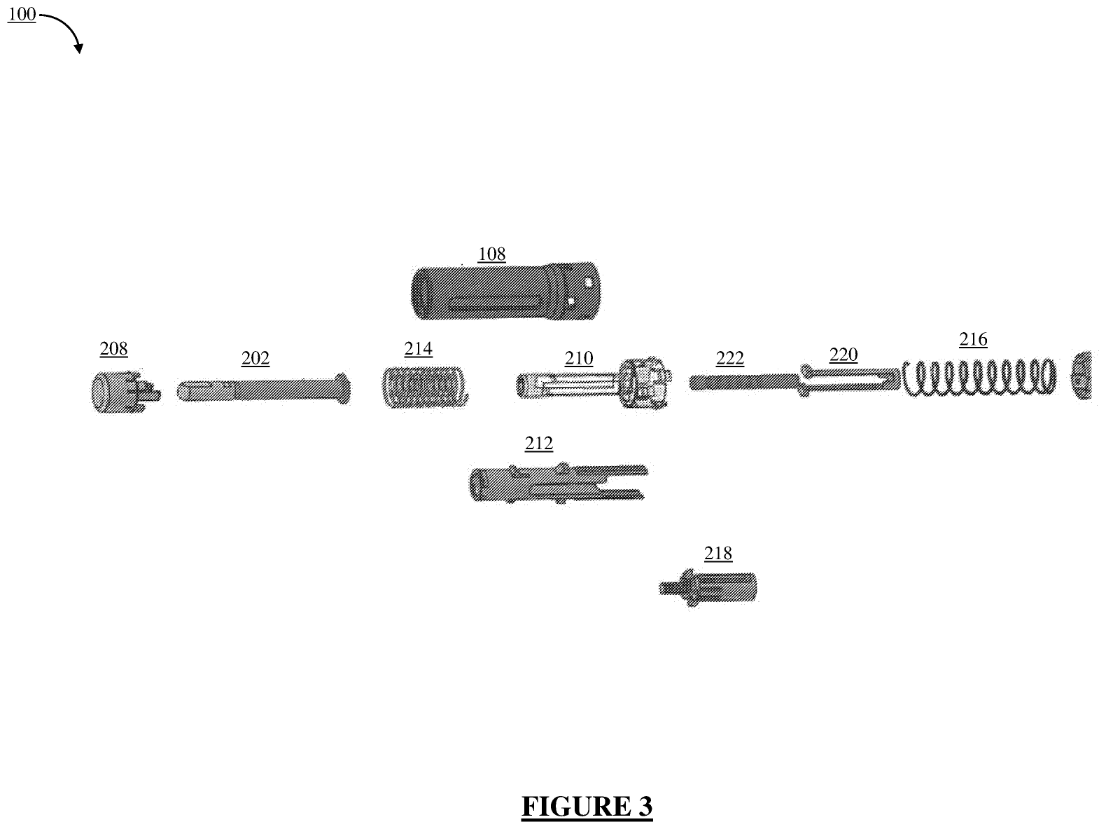

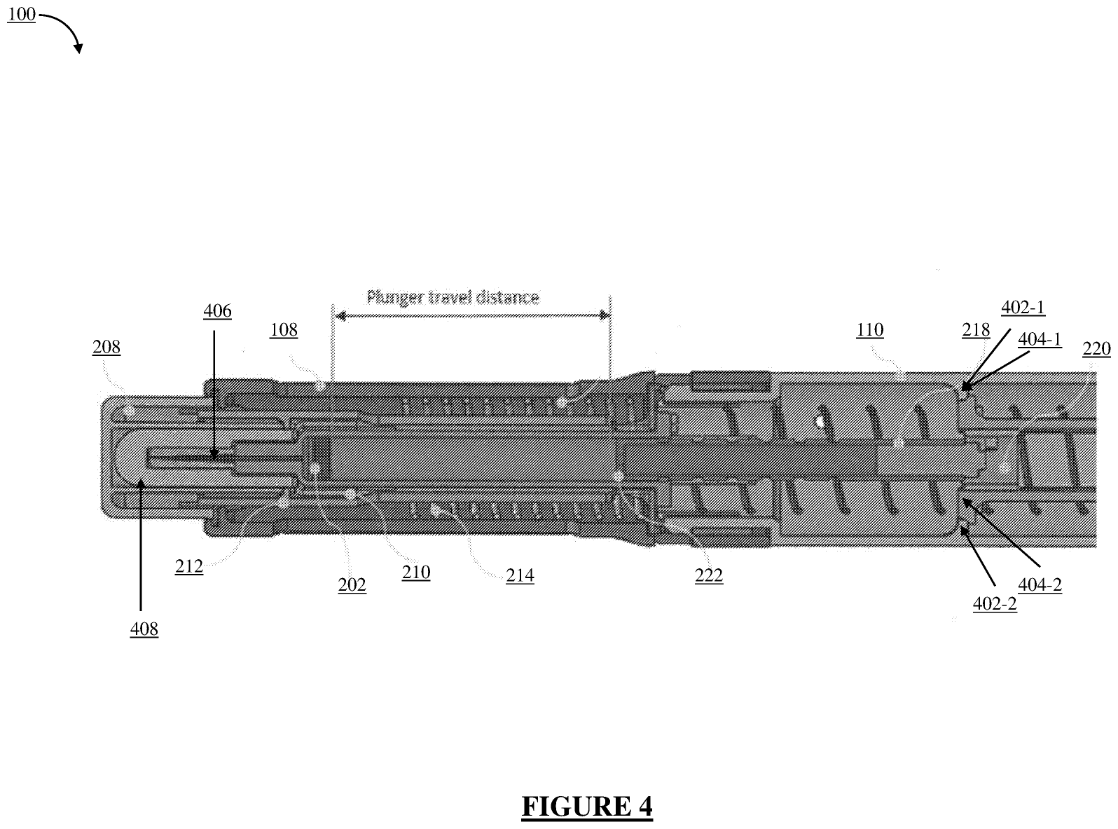

FIG. 1 illustrates a perspective view of an auto-injector assembly 100, according to an embodiment of the present disclosure. FIG. 2 illustrates an exploded view of the auto-injector assembly 100 depicting various constituent components, according to an embodiment of the present disclosure. FIG. 3 illustrates another exploded view of the auto-injector assembly 100, according to an embodiment of the present disclosure. FIG. 4 illustrates a sectional view of the auto-injector assembly 100 depicting various constituent components, according to an embodiment of the present disclosure. Referring to FIG. 1, FIG. 2, FIG. 3, and FIG. 4, the auto-injector assembly 100 may include, but is not limited to, a housing 102, a syringe assembly 202 having a needle 406, a needle shield 408, a cap 208, a syringe holder 210, a needle guard 212, a needle guard spring 214, a dose delivery spring 216, a spring holder 218, a locking unit 220, a plunger 222.

In an embodiment, the housing 102 may include a proximal portion 108 and a distal portion 110. The proximal portion 108 may accommodate the positioning of the syringe assembly 202, the needle shield 408, the cap 208, the syringe holder 210, the needle guard 212, and the needle guard spring 214. Further, the distal portion 110 may accommodate the dose delivery spring 216, the spring holder 218, the locking unit 220, and the plunger 222.

The housing 102 may include a proximal end 104 and a distal end 106. The syringe assembly 202 may be disposed adjacent to the proximal end 104. The syringe assembly 202 may be supported within the housing 102 through the syringe holder 210. The syringe assembly 202 may be adapted to store a predetermined volume of a drug to be administered. The syringe assembly 202 may include the needle 406 through which the drug is administered into the injection site.

In an embodiment, the needle shield 408 may be adapted to encapsulate the needle 406. Further, the needle shield 408 may be fixed with the cap 208, which may be adapted to close the proximal end 104 of the housing 102 when not in use. The cap 208 and the needle shield 408 may be adapted to be removed before initiating the process of administering of the drug.

Further, the needle guard 212 may be disposed to surround the syringe assembly 202. The needle guard 212 may be positioned such that a portion of the needle guard 212 is adapted to extend out of the housing 102 in an initial state. The auto-injector assembly 100 is activated by the user, for example, by pressing the proximal end 104 on the injection site. When the extended portion is pressed on to an injection site, the needle guard 212 may be adapted to retract into the housing 102. In this process, the needle 406 of the syringe assembly 202 is extended for being inserted into the injection site and to return to an original position when the drug is administered.

In an embodiment, the needle guard spring 214 may be adapted to support the movement of the needle guard 212 within the housing 102 for administering the drug. The needle guard spring 214 may be adapted to be disposed between an inner surface of the housing 102 and an outer surface of the needle guard 212. Therefore, the space between the housing 102 and the needle guard 212 accommodates the movement, i.e., compression and expansion, of the needle guard spring 214. The needle guard spring 214 may accommodate the extension and retraction of the needle guard 212 within the housing 102 during the administering of the drug.

The auto-injector assembly 100 may further include the spring holder 218 disposed adjacent to the distal end 106 of the housing 102. The spring holder 218 may be adapted to hold the dose delivery spring 216 in a compressed state. The dose delivery spring 216 may be disposed between the inner surface of the housing 102 and an outer surface of the spring holder 218. Such positioning of the dose delivery spring 216 outside the plunger 222 allows accommodating drugs having higher viscosity, for example, by changing spring stiffness, say, by increasing wire diameters or an overall spring diameter. In another embodiment, the dose delivery spring 216 may be disposed inside the spring holder 218.

The spring holder 218 may be adapted to be in a locked state with the housing 102. In an embodiment, the locking unit 220 may be disposed inside the spring holder 218 and adapted to keep the spring holder 218 in the locked state with the housing 102.

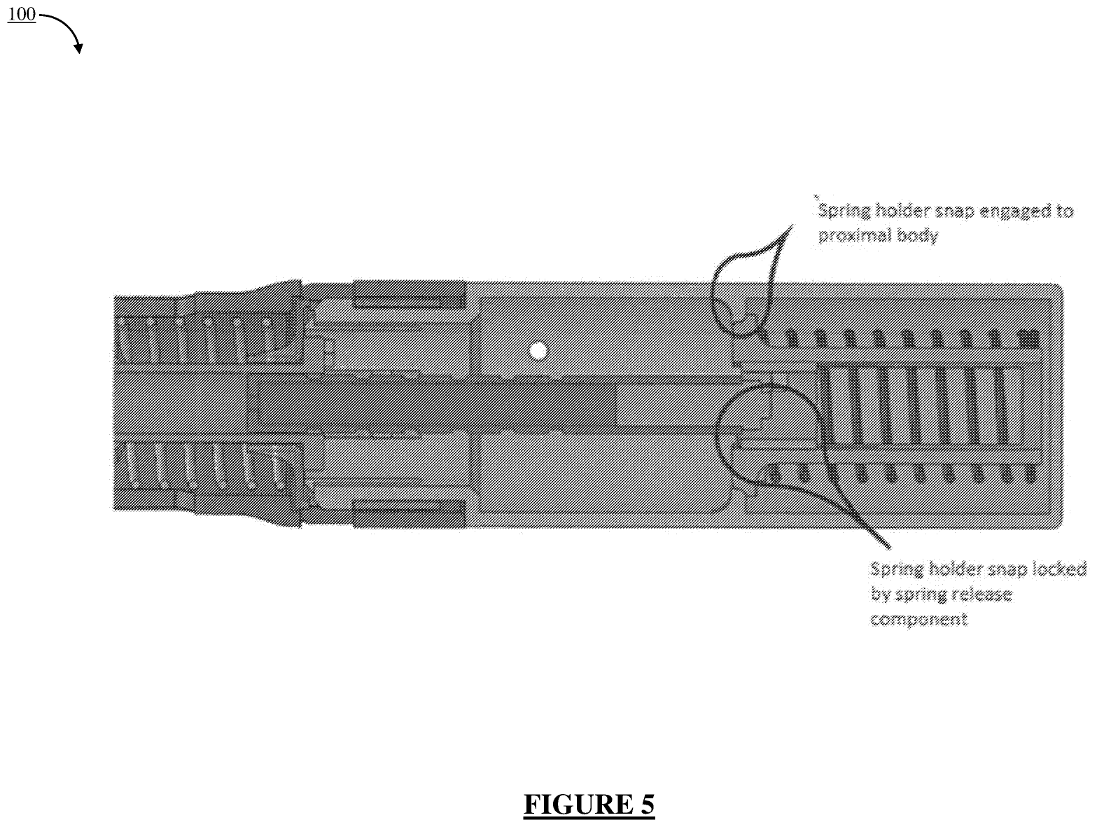

FIG. 5 illustrates a sectional view of a portion of the auto-injector assembly 100 depicting locking of the spring holder 218 with the housing 102, according to an embodiment of the present disclosure. Referring to FIG. 4 and FIG. 5, for locking the spring holder 218, the housing 102 may include a pair of stoppers 402 formed on the inner surface. Further, the spring holder 218 may include a pair of arms 404 adapted to engage with the pair of stoppers 402 for locking the spring holder 218 with the housing 102.

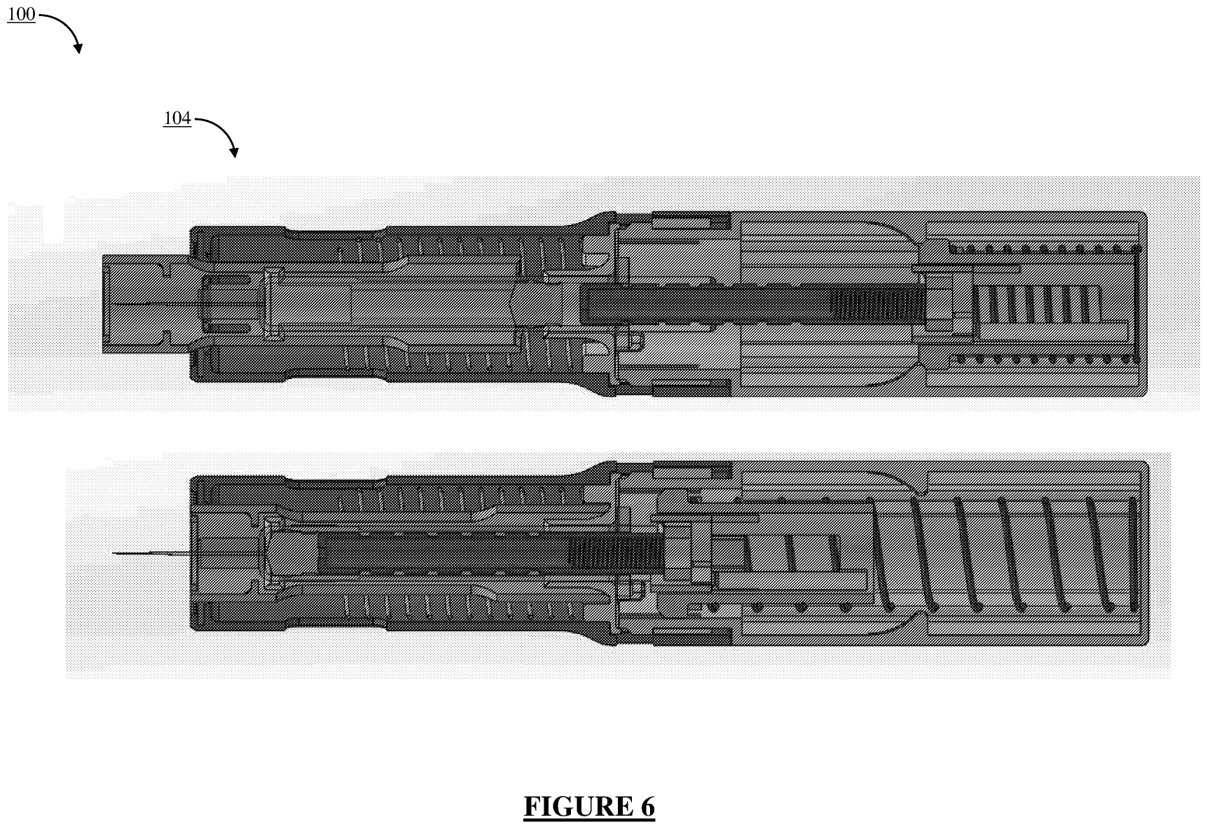

Further, the spring holder 218 may be engaged with the locking unit 220 as well. For example, the spring holder 218 may be snap-locked with the locking unit 220. In an embodiment, the needle guard 212, when being retracted, may be adapted to push the locking unit 220 further into the spring holder 218 thereby unlocking the spring holder 218 from the housing 102. FIG. 6 illustrates a sectional view of the auto-injector assembly 100 depicting unlocking of the spring holder 218 owing to movement of the needle guard 212, according to an embodiment of the present disclosure.

The spring holder 218 is unlocked such that the plunger 222 is moved towards the syringe assembly 202 for administering the drug into the injection site. In an embodiment, the spring holder 218 may be adapted to unlock, releasing the dose delivery spring 216 from the compressed state and pushing the plunger 222 towards the syringe assembly 202 based on a compressive force of the dose delivery spring 216. Therefore, the plunger 222 may be adapted to move towards the syringe assembly 202 when the spring holder 218 is unlocked. In the locked state of the spring holder 218, the movement of the plunger 222 is restricted.

The plunger 222 may be engaged with the spring holder 218. In an embodiment, the plunger 222 may include a plurality of threads formed on an outer surface. Further, the spring holder 218 may include a nut (not shown) adapted to engage with the plurality of threads of the plunger 222. The plunger 222 may be rotated into the nut for controlling the travelling distance of the plunger 222 within the housing 102 to administer the drug into the injection site based on the predetermined volume of the drug in the syringe assembly 202.

In another embodiment, the plunger 222 may include a plurality of threads formed on an inner surface. Further, the spring holder 218 may include a shaft 224 adapted to engage with the plurality of threads of the plunger 222. The plunger 222 may be adapted to rotate into the shaft 224 for controlling the travelling distance of the plunger 222 within the housing 102 to administer the drug.

Therefore, a position of engagement of the plunger 222 with the spring holder 218 may be adapted to change for controlling a travelling distance of the plunger 222 within the housing 102 based on the predetermined volume of the drug in the syringe assembly 202.

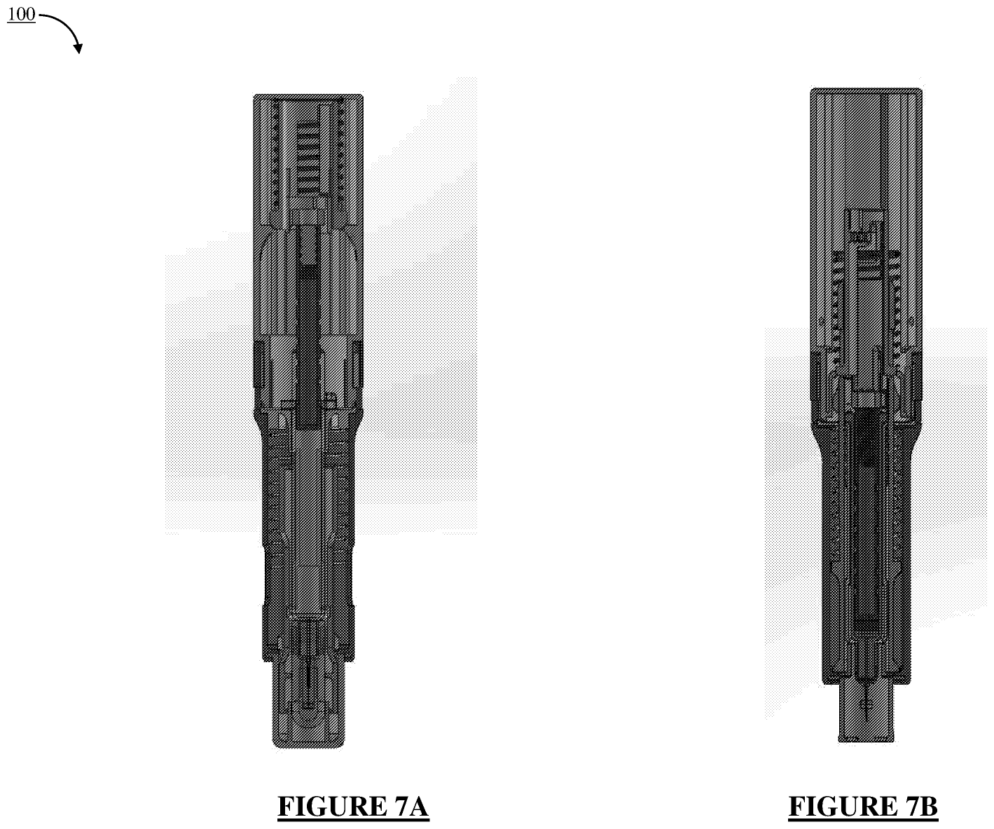

FIG. 7A illustrates a sectional view of the auto-injector assembly 100 depicting position of constituent components in an initial state, i.e., before the needle guard 212 is pressed on the injection site, according to an embodiment of the present disclosure. Further, FIG. 7B illustrates a sectional view of the auto-injector assembly 100 depicting position of constituent components after drug is administered, according to an embodiment of the present disclosure. As illustrated, the plunger 222 along with the spring holder 218 moves towards the syringe assembly 202 to push the drug through the needle 406 into the injection site.

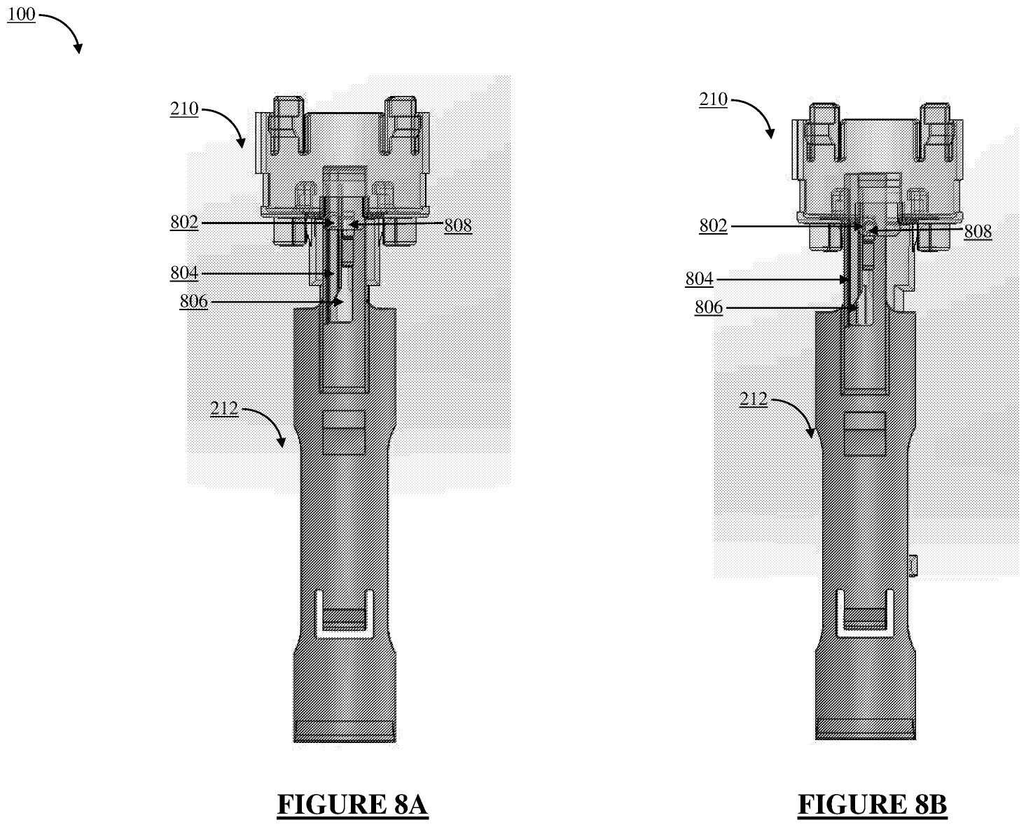

In an embodiment, the auto-injector assembly 100 may include a provision to ensure that the device is not reused. Particularly, the engagement of the needle guard 212 and the syringe holder 210 during the retraction and extension is such that the needle guard 212 is locked with the syringe holder 210, ensuring to restrict further movement of the needle guard 212. FIG. 8A illustrates positioning of the needle guard 212 with respect to the syringe holder 210 in the initial state, according to an embodiment of the present disclosure. The syringe holder 210 may include a lock portion 802 formed on an outer surface. Further, the needle guard 212 may include, but is not limited to, a longitudinal groove 804 adapted to receive the lock portion 802 of the syringe holder 210.

The lock portion 802 may be adapted to travel along the longitudinal groove 804 for accommodating relative movement of the needle guard 212 with respect to the syringe holder 210 when the needle guard 212 retracts into the housing 102. Further, the needle guard 212 may include a longitudinal slot 806 formed adjacent to the longitudinal groove 804. The longitudinal slot 806 may be adapted to accommodate return travel of the lock portion 802 when the needle guard 212 is moving back to the original position after administering of the drug.

FIG. 8B illustrates positioning of the needle guard 212 with respect to the syringe holder 210 after the drug is administered, according to an embodiment of the present disclosure. As illustrated, the longitudinal slot 806 may include a stopper slot 808 that is adapted to hold the lock portion 802 at the end of return travel, restricting further movement of the needle guard 212 once the drug is administered. This provision of the auto-injector assembly 100 acts as a safety feature to avoid needle stick injuries, for example, after the disposal of the auto-injector assembly 100.

In an embodiment, the auto-injector assembly 100 may include a viewing slot 112 formed on the housing 102 to view the movement of the plunger 222 from outside. Therefore, the user can inspect right usage of the auto-injector assembly 100 from the outside.

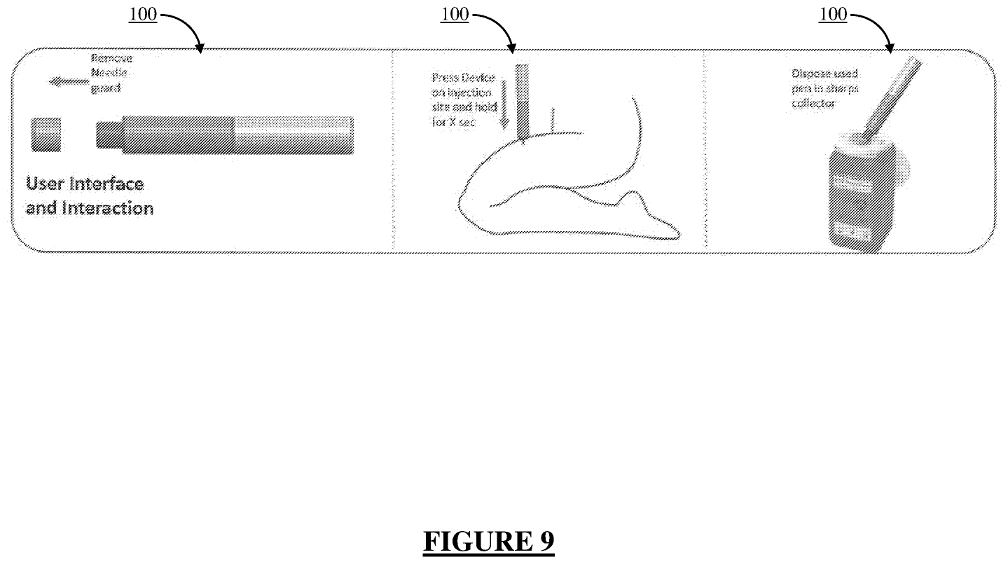

FIG. 9 illustrates an example depicting usage of the auto-injector assembly 100 by a user, according to an embodiment of the present disclosure. As shown, the cap 208 is removed. The auto-injector assembly 100 is then positioned on an injection site on the patient and held there for a predefined time duration. The auto-injector assembly 100 is activated by pressing the needle guard 212 on the injection site. The pressing of the needle guard 212 may activate needle insertion and dose dispensing with one action by the patient. Particularly, after removing the cap 208, the user would press the proximal end 104 of the auto-injector assembly 100, i.e., the extended portion of the needle guard 212 on to the injection site. Once the drug is delivered, used pens may be disposed in a sharps collector.

As would be gathered, the auto-injector assembly 100 of the present disclosure offers a comprehensive and effective approach for delivering drugs in varying volumes into the patient's body. In operation, the user removes the cap 208 along with the needle shield 408, before positioning the auto-injector assembly 100 on the injection site. The needle guard 212 is then pressed on the injection site. Consequently, the needle guard 212 is retracted into the housing 102, pushing the locking unit 220 and unlocking the spring holder 218. Consequently, owing to the expansion of the dose delivery spring 216, the spring holder 218 along with the plunger 222 moves towards the syringe assembly 202 to push the drug into the injection site through the needle 406. Further, the needle guard 212 while reaching the original position, due to the expansion of the needle guard spring 214, locks with the syringe holder 210, restricting any further movement.

Once the delivery of drug is complete and the auto-injector assembly 100 is removed, the needle guard 212 springs back and returns to its initial position covering the needle 406. This protects the user from needle stick injury.

Therefore, the auto-injector assembly 100 is a drug delivery mechanism that can be used with various kinds of drugs requiring to be delivered to the patient through a prefilled syringe or a cartridge. The construction of the auto-injector assembly 100 is such that only one such auto-injector assembly 100 can be used for different injection requirements by varying the operating settings.

The plunger 222 can be engaged at different positions with the spring holder 218, for example, through the nut or the shaft 224, based on different volumes of the drug to be administer by the auto-injector assembly 100. Further, the spring holder 218 is locked with the housing 102 through the engagement of the arms 404 and the stoppers 402. Therefore, the spring holder 218 and the plunger 222 cannot move unless being unlocked by the needle guard 212. Moreover, once the drug is administered, the needle guard 212 locks with the syringe holder 210 to ensure that the auto-injector assembly 100 is not reused.

Owing to the same device being used for different drug volume requirements, this would significantly reduce manufacturing cost and inconvenience for a manufacturer. Further, an overall operational cost of the auto-injector assembly 100 is also reduced. Further, handling and maintenance of multiple auto-injectors, as is the case in the existing techniques, are significantly reduced. Therefore, the auto-injector assembly 100 of the present disclosure is easy to use, cost-effective, compact, and operation-effective, and offers an ease of manufacturing, an ease of maintenance, and an ease of operation.

The auto-injector assembly 100 of the present disclosure may be used for delivering different therapeutic compounds, such as drugs and biologics, including but not limited to, antibodies, antisense, RNA interference, gene therapy, primary and embryonic stem cells, vaccines, and combinations thereof. For instance, the embodiments described herein may be utilized in combination with known monoclonal antibodies including, but not limited to, Abciximab, Abituzumab, Abrilumab, Actoxumab, Adalimumab, Adecatumumab, Aducanumab, Afasevikumab, Afelimomab, Afutuzumab, Alacizumab pegol, ALD518, ALD403, Alemtuzumab, Alirocumab, Altumomab pentetate, Amatuximab, AMG 334, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab, Atorolimumab, Avelumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Canakinumab, Cantuzumab mertansine, Cantuzumab ravtansine, Caplacizumab, Capromab pendetide, Casirivimab, Carlumab, Carotuximab, Catumaxomab, cBR96-doxorubicin immunoconjugate, Cedelizumab, Cergutuzumab amunaleukin, Certolizumab pegol, Cetuximab, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Crotedumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dinutuximab, Diridavumab, Domagrozumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epratuzumab, Erenumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Fibatuzumab, Ficlatuzumab, Figitumumab, Firivumab, Flanvotumab, Fletikumab, Fontolizumab, Foralumab, Foravirumab, Fresolimumab, Fulranumab, Futuximab, Galcanezumab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Idarucizumab, Igovomab, IMA-638, IMAB362, Imalumab, Imciromab, Imdevimab, Imgatuzumab, Inclacumab, Indatuximab ravtansine, Indusatumab vedotin, Inebilizumab, Infliximab, Inolimomab, Inotuzumab ozogamicin, Intetumumab, Ipilimumab, Iratumumab, Isatuximab, Itolizumab, Ixekizumab, Keliximab, Labetuzumab, Lambrolizumab, Lampalizumab, Lanadelumab, Landogrozumab, Laprituximab emtansine, LBR-101/PF0442g7429, Lebrikizumab, Lemalesomab, Lendalizumab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, LY2951742, Mapatumumab, Margetuximab, Maslimomab, Matuzumab, Mavrilimumab, Mepolizumab, Metelimumab, Milatuzumab, Minretumomab, Mirvetuximab soravtansine, Mitumomab, Mogamulizumab, Monalizumab, Morolimumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Prezalizumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Risankizumab, Rituximab, Rivabazumab pegol, Robatumumab, Roledumab, Romosozumab, Rontalizumab, Rovalpituzumab tesirine, Rovelizumab, Ruplizumab, Sacituzumab govitecan, Samalizumab, Sapelizumab, Sarilumab, Satumomab pendetide, Secukinumab, Seribantumab, Setoxaximab, Sevirumab, SGN-CD19A, SGN-CD33A, Sibrotuzumab, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tamtuvetmab, Tanezumab, Taplitumomab paptox, Tarextumab, Tefibazumab, Telimomab aritox, Tenatumomab, Teneliximab, Teplizumab, Teprotumumab, Tesidolumab, Tetulomab, Tezepelumab, TGN1412, Ticilimumab, Tigatuzumab, Tildrakizumab, Timolumab, Tisotumab vedotin, TNX-650, Tocilizumab, Toralizumab, Tosatoxumab, Tositumomab, Tovetumab, Tralokinumab, Trastuzumab, Trastuzumab emtansine, TRB S07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Vobarilizumab, Volociximab, Vorsetuzumab mafodotin, Votumumab, Xentuzumab, Zalutumumab, Zanolimumab, Zatuximab, Ziralimumab, and Zolimomab aritox or combinations thereof.

While specific language has been used to describe the present subject matter, any limitations arising on account thereto, are not intended. As would be apparent to a person in the art, various working modifications may be made to the method in order to implement the inventive concept as taught herein. The drawings and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment.

1-10. (canceled)

11. An auto-injector assembly comprising:
a housing having a proximal end and a distal end; a syringe assembly disposed adjacent to the proximal end of the housing and adapted to store a predetermined volume of a drug to be administered through a needle;
a needle guard disposed to surround the syringe assembly such that a portion of the needle guard is adapted to extend out of the housing in an initial state and to retract into the housing when the portion is pressed on to an injection site;
a spring holder disposed adjacent to the distal end of the housing and adapted to hold a dose delivery spring in a compressed state;
a locking unit disposed inside the spring holder and adapted to keep the spring holder in a locked state with the housing; and
a plunger engaged with the spring holder and adapted to move towards the syringe assembly when the spring holder is unlocked, wherein a position of engagement of the plunger with the spring holder is adapted to change for controlling a travelling distance of the plunger within the housing based on the predetermined volume of the drug in the syringe assembly,
wherein the needle guard, when being retracted, is adapted to push the locking unit further into the spring holder, unlocking the spring holder from the housing such that the plunger is moved towards the syringe assembly for administering the drug into the injection site.

12. The auto-injector assembly as claimed in claim 11, wherein the needle guard is adapted to be pressed against the injection site to extend the needle of the syringe assembly for being inserted into the injection site and to return to an original position when the drug is administered.

13. The auto-injector assembly as claimed in claim 11, comprising a syringe holder adapted to support the syringe assembly within the housing; and a needle guard spring adapted to be disposed between an inner surface of the housing and an outer surface of the needle guard, wherein the needle guard spring is adapted to support the movement of the needle guard within the housing for administering the drug.

14. The auto-injector as claimed in claim 11, comprising: the housing having a pair of stoppers formed on an inner surface; and the spring holder comprising a pair of arms adapted to engage with the pair of stoppers for locking the spring holder with the housing, restricting the movement of the plunger.

15. The auto-injector as claimed in claim 11, comprising: the plunger having a plurality of threads formed on an outer surface; and the spring holder comprising a nut adapted to engage with the plurality of threads of the plunger, wherein the plunger is rotated into the nut for controlling the travelling distance of the plunger within the housing to administer the drug into the injection site based on the predetermined volume of the drug in the syringe assembly.

16. The auto-injector as claimed in claim 11, comprising: the plunger having a plurality of threads formed on an inner surface; and the spring holder comprising a shaft adapted to engage with the plurality of threads of the plunger, wherein the plunger is rotated into the shaft for controlling the travelling distance of the plunger within the housing to administer the drug into the injection site based on the predetermined volume of the drug in the syringe assembly.

17. The auto-injector assembly as claimed in claim 11, wherein the spring holder is adapted to unlock, releasing the dose delivery spring from the compressed state and pushing the plunger towards the syringe assembly based on a compressive force of the dose delivery spring.

18. The auto-injector assembly as claimed in claim 11, comprising: a needle shield adapted to encapsulate the needle; and a cap fixed with the needle shield and adapted to close the proximal end of the housing when not in use, wherein the cap and the needle shield are adapted to be removed before pressing the needle guard against the injection site for administering the drug.

19. The auto-injector assembly as claimed in claim 13, comprising: the syringe holder having a lock portion formed on an outer surface; and the needle guard comprising: a longitudinal groove adapted to receive the lock portion of the syringe holder, wherein the lock portion is adapted to travel along the longitudinal groove for accommodating relative movement of the needle guard with respect to the syringe holder when the needle guard retracts into the housing; and a longitudinal slot formed adjacent to the longitudinal groove and adapted to accommodate return travel of the lock portion when the needle guard is moving back to the original position after administering of the drug, wherein the longitudinal slot comprising a stopper slot that is adapted to hold the lock portion at the end of return travel, restricting further movement of the needle guard once the drug is administered.

20. The auto-injector assembly as claimed in claim 11, comprising a viewing slot formed on the housing to view the movement of the plunger from outside.
